(19) United States
(12) Patent Application Publication
(10) Pub. No.: US 2018/0168925 A1
(43) Pub. Date: Jun. 21, 2018

(54) METHOD OF MEDICAL TREATMENT OF A PATIENT UNDER TOTAL SUBMERGENCE IN WATER COMPLETELY SURROUNDING AND AFFECTING THE PATIENT'S ORGANISM

(71) Applicant: Krzysztof D. WOZNIAK, Vien (AT)

(72) Inventor: Krzysztof D. WOZNIAK, Vien (AT)

(21) Appl. No.: 15/737,063

(22) PCT Filed: Jun. 16, 2016
(86) PCT No.: PCT/IB2016/000844

§ 371 Date: Dec. 15, 2017

(30) Foreign Application Priority Data
Jun. 17, 2015 (PL) P.412755

(51) Int. Cl. A61H 33/00; A61G 10/02; A61M 16/10; A61M 16/12

(52) U.S. Cl. CPC A61H 33/0087; A61H 33/0095; A61H 33/6005; A61G 10/026; A61M 16/1005; A61M 2202/0208; A61H 2201/0207; A61H 2201/0214; A61H 2201/107; A61H 2201/1261; A61H 2203/02; A61M 16/125

(57) ABSTRACT

The invention relates to a medical use of water exerting a hydrostatic pressure higher than 1.1 bar, completely surrounding and affecting a patient's organism under total submergence, for use in prophylaxis, physiotherapy, rehabilitation and therapy of disorders, defects and injuries of skeletal system, arthroskeletal system, musculoskeletal system and other body systems such as circulatory system, nervous system, immune system, as well as pain conditions, such as migraine, myalgia and neuralgia, skin diseases, fertility disorders, potency dysfunctions, as well as overweight, obesity and defects of posture. According to the invention the hydrostatic pressure of water is preferably higher than 1.2 bar, and the patient is human being. The invention is of a particular importance in rehabilitation and therapy that follows after aesthetic medicine treatment, after surgeries, after physical injuries of motor system. The solution according to the invention is advantageously used in combination with procedures of classic hydrotherapy, manual therapy, kinesitherapy or combination thereof. It is of a particular benefit to provide air, mixture of gases of a composition different to the composition of air, mixtures of air and oxygen or pure oxygen for breathing (hyperbaric oxygen therapy HBO/HBOT), while the water may be supplemented with various additives appropriate for the kind of an ailment treated, such as mineral, gaseous, herbal, antiinflammatory, antimicrobial including antibacterial additives.

### FIELD OF THE INVENTION

The invention relates to a medical use of water completely surrounding, exerting a hydrostatic pressure and affecting a patient's organism under total submergence for a period of time longer than duration of breath holding, using an apparatus allowing breathing under the water. In particular the invention relates to the use of water exerting a hydrostatic pressure, completely surrounding and affecting a patient's organism under total submergence in prophylaxis, physiotherapy, rehabilitation and therapy of medical conditions adversely affecting skeletal system, arthroskeletal system, musculoskeletal system, nervous system and other body systems such as circulatory system, immune system, as well as in the therapy of selected skin diseases and internal diseases. Within the framework of a broad meaning of prophylaxis, the invention is foreseen for use in recreation and leisure, as well.

### PRIOR ART

Hydrotherapy has a very long tradition. Water and mud bathes, hydromasage, water whips have earned a deserved reputation among a medical personnel (staff) and are highly appreciated for their effectiveness by the users. Hydrotherapeutic procedures are prescribes in many medical indications and usually consist of a series of several to over a dozen of individual sessions, each one lasting a quarter to half or one hour. Repeating certain types of treatment at least once a year is recommended for patients suffering from defects and degenerative changes of skeletal system and from chronic forms of diseases affecting arthroskeletal system, musculoskeletal system, circulation system or other body systems such as for example nervous system. In case of intensive rehabilitation procedures prescribed after surgeries, after traffic accidents or other events of invasive character, series of the procedures are repeated until physical functions are regained to the extent prevailing before that event.

Balneotherapy, including salt bath, mud bath, peloid bath, sulphide bath, is applied to the whole body of a patient, however not under total submergence in the bath media (in corresponding bathing tabs), or exclusively to a selected organ such as lower or upper limbs (legs, arms) in small bathtubs of a shape adjusted to the type of the limb. The procedures may include alternative temperature bathing: hot bath alternately with cold bath. In cosmetic and embellishment use such bath treatments are used in face, neck, back and decolletage skin treatments.

It is also known to use prophylactically exercises in natural and artificial water reservoirs under apparent loss in a body weight resulting from the Archimedes buoyancy principle. Such exercises may take a form of activities performed in water individually or in a group such as aerobics in water. Also swimming, as a sport that symmetrically activates all groups of body musculature and all the limbs, is a recommended form of prophylaxis of diseases and defects of spinal column and musculoskeletal system. Swimming is an invaluable form of rehabilitation of disabled people with defects of musculoskeletal system being either congenial malformations or acquired defects. It is also a popular form of recreation.

In warm climatic zones classic hydrotherapy may be intensified by introducing contact of a patient experiencing the therapy with aquatic animals, in particular dolphins and turtles.

Water whips are recognized as physiotherapeutic procedure of a very wide spectrum of activity, used to enhance functioning of a number of internal body organs and to eliminate uncontrolled strains and tensions in patients' organisms. The essence of this treatment is a local stimulation of receptors on the body surface and subcutaneous receptors, activated also by other ancient therapeutic techniques such as acupuncture and acupressure. Water whip is a stream of water leaving a nozzle under a significant pressure, the stream being directed towards selected parts of patient's body. Water temperature in the stream of water whip may range from hot through warm to cold. Alternating temperature of water whip is preferred.

Also known is a prophylactic and therapeutic treatment of patient's body in hyperbaric chambers filled with the air, being pumped to the chamber under the press sure of ca. 1.5 bar, wherein patients wearing gas masks breath pure oxygen, air enriched with oxygen or the air. Such a hyperbaric chamber is used among others in treatment of hard healing wounds and wide spread burns, chronic osteitis, bacterial tissue infections. Using this type of treatment is recommended in case of patients poisoned with carbon monoxide.

Thus far, deep water environment was not regarded as being advantageous for organisms of humans and was considered exclusively as a hostile one, its hostility being illustrated by diseases associated with work of underwater divers operating on large depths and by injuries and degradation of functions of organisms of professional divers diving without protective equipment—such as pearl divers in tropical regions of the seas.

Also free diving and scuba diving are the extreme sports, demanding good training and excellent physical fitness, as well as reliable technical equipment allowing safe diving in water down to large depths.

### SUMMARY OF THE INVENTION

It has now been unexpectedly found out that under controlled conditions beneficial effects of water exerting a hydrostatic pressure when completely surrounding and affecting a patient's organism under total submergence are observed.

It is an aim of the present invention to provide a novel prophylactic, physiotherapeutic, rehabilitation and therapeutic use of affecting body of a patient—in need of such a treatment, by an aquatic environment, wherein water exerts hydrostatic pressure and completely surrounds a patient's organism.

The present invention relates to water exerting a hydrostatic pressure higher than 1.1 bar, completely surrounding and affecting a patient's organism under total submergence, for use in medicine.

Water exerting a hydrostatic pressure higher than 1.1 bar, completely surrounding and affecting a patient's organism under total submergence, according to the invention is foreseen for use in prophylaxis, physiotherapy, rehabilitation and therapy of disorders, defects and injuries of skeletal system, arthroskeletal system, musculoskeletal system and other body systems such as circulatory system, nervous system, immune system.

Water exerting a hydrostatic pressure higher than 1.1 bar, completely surrounding and affecting a patient's organism under total submergence, according to the invention is foreseen for use in prophylaxis, physiotherapy, rehabilitation and therapy of pain conditions, such as migraine, myalgia and neuralgia, as well as in after aesthetic medical treatments, after surgeries, after physical injuries of motor system.

Water exerting a hydrostatic pressure higher than 1.1 bar, completely surrounding and affecting a patient's organism under total submergence, according to the invention is foreseen for use in prophylaxis and therapy of skin diseases, fertility disorders, potency dysfunctions, as well as in prophylaxis, physiotherapy, rehabilitation and therapy of overweight, obesity and defects of posture.

According to the present invention the patient is a human being and the hydrostatic pressure is preferably higher than 1.2 bar.

The present invention allows for a use of water exerting a hydrostatic pressure higher than 1.1 bar, completely surrounding and affecting a patient's organism under total submergence, for carrying out prophylactic, physiotherapeutic, rehabilitation and therapeutic procedures aiming to prevent (counteract), alleviate and cure of disorders, defects and injuries of skeletal system, arthroskeletal system, musculoskeletal system and other body systems such as circulatory system, immune system, nervous system, as well as selected skin diseases and internal diseases, for the time and with frequency sufficient to attain desired prophylactic or therapeutic effect.

According to the present invention, together with the patient's total submergence, using the environment of water exerting a hydrostatic pressure higher than 1.1 bar, completely surrounding and affecting a patient's organism, preferably simultaneously procedures of classic hydrotherapy, manual therapy, kinesitherapy or combinations thereof are applied and used.

Advantageously, together with the patient's total submergence, using the environment of water exerting a hydrostatic pressure higher than 1.1 bar, completely surrounding and affecting a patient's organism, also local thermal stimuli are used, the stimuli being of temperatures higher or lower than patient's body temperature.

According to the present invention, during the patient's total submergence, using the environment of water exerting a hydrostatic pressure higher than 1.1 bar, completely surrounding and affecting a patient's organism, the air, mixture of gases of a composition different from the composition of air, mixtures of air and oxygen or pure oxygen (hyperbaric oxygen therapy HBO/HBOT) are used for breathing, whereas the water is supplemented with additives appropriate for the type of the patient's disorder, such as mineral, gaseous, herbal antiinflammatory, antimicrobial—including antibacterial, additives.

The invention may be reduced to practice in natural or artificial water reservoirs, under patient's total submergence, making use—in prophylaxis, physiotherapy, rehabilitation and therapy of the above mentioned diseases, injuries and defects, of hydrostatic pressure higher than 1.1 bar, exerted by water completely surrounding and affecting a patient's organism, while providing the patient and optionally also a therapist with a permanent or a transferable infrastructure for breathing the air, mixture of gases of a composition different from the composition of air, mixtures of the air and oxygen or pure oxygen, as well as with an infrastructure for activating skin senses with various stimuli, including thermal and electric stimuli. The reservoir may be an adapted or dedicated one, both for a single user and for more users, at the time.

The treatments in which solution according to the present invention is used, allow to carry out classic prophylactic, physiotherapeutic, rehabilitation and therapeutic procedures, under a patient's total submergence, making use of hydrostatic pressure higher than 1.1 bar, exerted by water completely surrounding and affecting the patient's organism.

An essential feature of the treatments according to the present invention is reduction of volume of soft tissues, such as for example intervertebral and articular tissues that have lower density in comparison to the hard tissues of skeletal system. Thanks to the phenomenon, patient's skeletal system becomes more loose in effect of gaining some extra free space within locations occupied under the normal pressure by the soft tissues. It results in an increased mobility of articular joints and simultaneous reduction of pain associated with their movement without administration of analgesics that are required when exercises take place in usual aerostatic environment (in the air and under the atmospheric pressure).

It thus creates an advantageous environment for therapies of various kinds, for example physiotherapy: manual therapy or kinesitherapy, of arthroskeletal system, musculoskeletal system or nervous system.

An additional rehabilitation or therapeutic effect may be attained if and when patient is provided with gas other than the air for breathing, i.e.—depending on the depth on which patient is submerged, with the air enriched with oxygen or pure oxygen, so as in hyperbaric oxygen therapy HBO/HBOT. Simultaneously bactericidal additives, such as ozone, may be added to the water surrounding the patient during the treatment, or else the water may be supplemented with preparations enhancing wound healing.

The environment of water exerting a hydrostatic pressure higher than 1.1 bar, completely surrounding and affecting a patient's organism under total submergence, eliminates stimuli originating from the external world and being simultaneously conducive to patient's physical activity. This is a very advantageous aspect in case of therapeutic treatment of nervous system disorders, such as autism, ADHD (attention deficit hyperactivity disorder), Alzheimer disease, Parkinson disease, impairment of physical activity in effect of stroke, or even depression. Total submergence in water bath alters perception of stimuli such as surrounding temperature and thermal stimuli applied locally, movements of ambient environment (still water or water streams of controlled intensity), visual and audio impressions.

Advantages of the present solution over the environment of known hyperbaric chambers should be emphasized. Under a neutral tare, in the present solution disadvantageous overloads typical for hyperbaric chambers filled with the air are eliminated. Additionally, in effect of gradual increase of the height of the water column above the patient's body due to its descending to a greater depth or due to increasing water level above the patient's body in artificial water reservoir, individually selected speed of accommodation of the patient's organism to an increased hydrostatic pressure is possible and can be adjusted accordingly.

Within the framework of a broad meaning of prophylaxis, the invention covers also recreational use of therapeutic means of the present invention, the means being water exerting a hydrostatic pressure higher than 1.1 bar, completely surrounding and affecting a patient's organism under total submergence, in underwater fitness exercises, gymnastics, running, rhythmical physical exercises analogous to water aerobics, under hydrostatic pressure exceeding 1.1 bar, preferably higher than 1.2 bar.

### SHORT DESCRIPTION OF DRAWING

The detail description provided below explains the present invention in more detail with reference to the accompanying drawing, wherein

FIG. 1 is a schematic side view of a construction and facilities provided in a water reservoir suitable for working the solution according to the invention, i.e. for carrying out prophylactic, physiotherapeutic, rehabilitation and therapeutic procedures, making use of the therapeutic means of the present invention.

### DETAIL DESCRIPTION OF THE INVENTION

A water environment completely surrounding organism of a human being is commonly recognized as an advantageous and safe environment appropriate for fetal period of life. It is demonstrated, for example by bigger and bigger popularity of parturition (childbirth) in water bath, early swimming training organized for newborn babies, popularity of water relaxing procedures and various types of spas enjoyed at home or in specialized therapeutic centers such as sanatoria, health spa and wellness cabinets.

The present invention extends beneficial effects of water environment performance to prophylactic, physiotherapeutic, rehabilitation and therapeutic procedures, by means of making use of water exerting a hydrostatic pressure higher than 1.1 bar, preferably 1.2 bar, and—in contrast to the procedures used thus far, completely surrounding and affecting a patient's organism under total submergence. The indicated hydrostatic pressure values prevail at depths greater than 1.0 m, preferably greater than 2.0 m.

Hydrostatic pressure of liquids within gravitational field depends solely on heigth of liquid column and liquid density according to an equation p=ρgh, wherein p is a hydrostatic pressure, ρ is liquid density, g is a gravitational acceleration constant and h is a heigth of liquid column expressed consequently in SI or cgs units. Hydrostatic pressure is directly proportional to the heigth of liquid column above the measurement point. A graph illustrating this relation has a form of a straight line and inclination of the line depends on density of the liquid. For water, value of ρ is 1 g/ml. Sea water of a high mineral salts content has the density ρ>1. Therefore, at the same depth below the surface of water, in the sea water the hydrostatic pressure is slightly different to the hydrostatic pressure in the fresh water reservoir.

Hydrostatic pressure affects the object, being a patient's organism in accordance to the present invention, from all sides and uniformly influences the patient's tissues, physiological systems and internal organs. Gradual patient's descending to a greater depth or gradual increase of the height of the water column above the patient's body results in smooth change of hydrostatic pressure value, thus allowing accommodation of the organism to the changing pressure conditions.

Typical syndroms of caisson disease, up till now justifying potential fears associated with conducting hydrotherapy under patient's total submergence in water, i.e. under hydrostatic pressure exerted on the patient's body higher than 1.1 bar, preferably 1.2 bar, can be avoided according to the present invention through observance of basic safety rules being mandatory in diving.

For total patient's submergence during procedures employing the solution according to the present invention, an infrastructure needs to be provided to deliver the air, other mixture of gases, or pure oxygen to allow easy breathing under the water throughout duration of the prophylactic, physiotherapeutic, rehabilitation or therapeutic procedure in the environment of water exerting a hydrostatic pressure higher than 1.1 bar, completely surrounding and affecting a patient's organism under total submergence. Such an infrastructure may be positioned and fixed at the treatment site, or it may be portable, separate for each individual descending down to the predetermined depth.

Additionally, also an infrastructure allowing local application of stimulating thermal stimuli of temperatures lower or higher than the patient's body temperature, for example in form of movable nozzles generating additional streams of water of temperatures higher or lower than the temperature of the bath itself. Width of the nozzles and pressure of water having its thermal parameters different than the thermal parameters of water surrounding the patient's body may be regulated in wide ranges depending solely on the patient's body parameters and the patient's health condition or the status of the disease advancement. Selection of the appropriate parameters' values will not create any problems to those practitioners skillful in traditional prophylaxis, physiotherapy, rehabilitation or therapy performed in aerostatic environment or—in a classic way, in water environment without total submergence of patient's body.

While observing appropriate safety measures, the present hydrotherapy employing water that exerts hydrostatic pressure higher than 1.1 bar, while completely surrounding and affecting a patient's organism under total submergence, in accordance with the present invention, may be enriched by local application of electric stimuli, for example such as affecting pain threshold after vertebral column injuries or motorium system injuries.

Generally, in the environment of water exerting a hydrostatic pressure higher than 1.1 bar, completely surrounding and affecting a patient's organism under total submergence, according to the present invention all the physiotherapeutic and rehabilitation procedures applied in traditional physiotherapy and rehabilitation (including hydrotherapy under atmospheric pressure) may also be applied, in full accordance with the diseases and condition of a patient.

For performing additional physiotherapeutic procedures such as manual therapy and local or general kinesitherapy in the environment of water exerting a hydrostatic pressure higher than 1.1 bar, completely surrounding and affecting a patient's organism under total submergence, interaction with a therapeutist is required. The therapeutist may assist the patient while staying in the same environment, or when performing procedures engaging use of fully or semi-automated equipment, the therapeutist may control the procedure and/or operation of the equipment while staying outside of the water environment, after his optional participation and help in proper initiation of the exercise by the patient.

The environment of water exerting a hydrostatic pressure higher than 1.1 bar, completely surrounding and affecting a patient's organism under total submergence, decreases the apparent weight of the patient's body, i.e. the body of the person receiving the treatment, or of the patient's individual limbs, thus making patient's cooperation with the therapeutist easier and—on the other hand, allowing the therapeutist to act with less effort for a longer time without increasing patient's discomfort and to the contrary in many cases lowering the painfulness of the treatment.

As it is described above, water exerting the beneficial hydrostatic pressure affects in different way different tissues of a patient's organism. Increase of pressure by means of increasing the depth at which the patient is located during the treatment causes a proportional decrease of volume of soft tissues such as for example intervertebral and articular tissues that have lower density in comparison to the hard tissues of skeletal system, thus making the patient's skeletal system more loose due to gaining some more of a free space.

Depending on the kind of disorder and patient's condition, using the solution according to the present invention leads to better results and better timing in comparison with the results of water treatment without total patient's submergence or with the results of treatment in aerostatic environment. For example, there is a significant increase of chances for alleviation of a so called spinal disc herniation (a medical condition of intervertebral discs), in compa-rison with analogeous treatments in aerostatic environment.

Hydrostatic pressure higher than 1.1 bar exerted by water completely surrounding and affecting a patient's organism under total submergence, changes the parameters of soft tissues to the greater extent than parameters of hard tissues of skeletal system. As regards soft tissues, a visible effect of procedures carried out in accordance with the invention is diminishing of the tissue swellings.

Similarly to hyperbaric therapy performed in hyperbaric chambers filled with the air, where breathing pure oxygen or the air enriched with oxygen brings an increased amount of oxygen to sick and anoxic patient's tissues and organs, in the environment of water exerting a hydrostatic pressure higher than 1.1 bar, preferably higher than 1.2 bar, completely surrounding and affecting a patient's organism under total submergence, in accordance to the present invention, providing the air, a mixture of gases of a composition different from the composition of air, mixtures of the air and oxygen or pure oxygen for breathing improves blood circulation, enhance defensive mechanisms supporting the patient's organism in combating infections, and thus increased supply of oxygen has also bactericidal and bacteriostatic effect, increases clearance of carbon monoxide and also improves performance of the organism in various respects.

It is necessary to properly select value of hydrostatic pressure to be exerted by water completely surrounding and affecting a patient's organism under total submergence, duration of an intended treatment and composition of gaseous mixture for breathing (with simultaneous observance of basic rules of diving and optionally rules of necessary decompression)—to a kind of prophylactic or therapeutic treatment, the selection being fully dependant on the patient's disorder and condition.

The results achieved due to breathing the air enriched with oxygen or pure oxygen in the environment of water exerting a hydrostatic pressure higher than 1.1 bar, advantageously higher than 1.2 bar, completely surrounding and affecting a patient's organism under total submergence, according to the present invention, may be enhanced by an appropriate selection of supportive additives to the water environment in agreement with the rules of classic hydrotherapy. It is a further aspect of the present invention that the presently proposed treatment is additionally supplemented with positive effects of physical exercises in an advantageous temperature of the water environment, water decreasing patient's apparent weight.

In view of the above, the present invention respectively modified by means of regulation of hydrostatic pressure—exerted by water completely surrounding and affecting a patient's organism under total submergence, preferably above 1.2 bar, by means of breathing oxygen by a patient, by means of supplementing the water with additives—alleviating or eliminating defined diseases, by means of regulation of water temperature, as well as by using water jets from the nozzles surrounding patient's body and also by means of kinesitherapy (physical exercises), may be recommended to many groups of patients suffering from:
vertebral column diseases, in which operation under decreased patient's apparent weight is beneficial and diseases of articular joints of a limited mobility;
nervous system diseases, such as depression, ADHD, autism, Alzheimer disease, Parkinson disease, poststroke medical conditions, low mood and decreased spirit, stress;
pain conditions, such as migraine, myalgia and neuralgia;
circulatory system diseases, such as sclerosis and hypertension;
general systemic disorders affecting body as a whole, such as autoimmune diseases, chronic inflammatory diseases;
medical conditions developed after aesthetic medical treatments, after surgeries, after physical injuries of motor system as well as those requiring increasing of muscles, tendons and ligaments elasticity;
skin diseases, such as psoriasis, urticaria, rash, erythema, acne, atopic dermatitis;
swelling of soft tissues;
fertility and potency dysfunctions;
overweight, obesity and body deformities such as defects of posture, lack of slenderness, excess of fatty tissue, cellulite, both weaken and overly tense muscles;
   as well as in prophylaxis of the above indicated disorders and undesired medical conditions of organisms.

The term “prophylaxis” in the present description has a broad meaning and—beside the usual prevention of development of specific diseases, to which a patient is susceptible or genetically determinated, it also encompasses activities associated with a philosophy of “living a healthy life”, which covers various physical exercises, like fitness training, activities improving performance of the organism and strengthening the same, restoring one's viability and mental balance, as well as those positively affecting one's mood due to releasing tensions and stress. In this sense, making a prophylactic use of the solution according to the invention, leads to slowing down the processes of ageing, supports embellishing treatments and treatments that modulate one's figure, improve flexibility of one's body, reduce swellings—including swellings of one's legs, help to correct one's posture and in general adds to the quality of one's life.

It is also a new feature of the solution according to the present invention that the environment surrounding a patient during the treatment is so radically changed in comparison to prior art techniques. Static contact of a whole body with water, optionally supplemented with stimuli known from classic hydrotherapy, provides a patient with novel sensory sensations, brings the patient into nearly weightlessness state, enhances usual audio stimuli, ensures changed visual stimuli that favour mental isolation, meditation and concentration. Still further sensual feeling may be provided by thermal and electric stimuli, as well as by movements of water environment. The patient may experience activity of water currents and streams of a predetermined temperatures, or else may be floated in the stream of a speed selected according to the patient's weight. Additionally, physical exercises may be performed with the therapeutist's assistance or patient may exercise on his or her own and the exercises may consist of swimming both breaststroke and backstroke in a counter-stream of water pumped under increased pressure through nozzles located at the level of patient′ body.

Essentially, according to the presently disclosed solution in its prophylactic, physiotherapeutic, rehabilitation or therapeutic aspects, influencing a patient's organism by water completely surrounding patient's body under total submergence, exerting a hydrostatic pressure within the selected range, especially pressure higher than 1.1 bar, preferably higher than 1.2 bar, is not anyhow limited by the patient's age, fitness, agility or ability to communicate with others, because patients may participate in the treatments in the presence of accompanying person—other than therapeutist, who shall assist the patient in using an underwater breathing equipment. Such a not self-reliant person could be a chilled, an elderly person, an autistic patient, or else a patient suffering from an advanced Alzheimer disease. The accompanying person, however, has to use the same underwater breathing equipment.

It is possible to apply the solution according to the present invention in its aspects related to prophylactic, physiotherapeutic, rehabilitation or therapeutic use of water exerting a hydrostatic pressure higher than 1.1 bar, completely surrounding and affecting a patient's organism under total submergence, in all available natural and artificial water reservoirs provided with an equipment (infrastructure) enabling breathing under the water and with an infrastructure enabling application to patient's body of additional thermal or other kind of stimuli affecting patient's senses.

The existing natural and artificial water reservoirs may be easily adapted to meet the needs related to performing treatment according to the present invention in the environment of water exerting a hydrostatic pressure higher than 1.1 bar, completely surrounding and affecting a patient's organism under total submergence. The simplest adaptation scheme consists of providing a patient and a therapeutist with equipment for breathing under the water and with ballast means counteracting the Archimedes buoyancy of water and thus enabling them to stay for a long time at the predetermined depth in either horizontal or vertical positions. Additional facilities may include also lighting equipment. The treatment sessions may be (film- or video-) recorded in order to provide medical consultants and/or doctor ordering the treatment with complete information on the course of performed prophylaxis, physiotherapy, rehabilitation or therapy procedures.

Even the most common swimming pools, not more than 1.3 m deep, may be used to perform treatment employing water exerting a hydrostatic pressure higher than 1.1 bar, completely surrounding and affecting a patient's organism under total submergence, at the bottom of such a swimming pool, where a patient is in a horizontal position and the therapeutist accompanies and assists the patient.

Wider ranges of hydrostatic pressures affecting a patient's organism under total submergence are available in the existing natural water reservoirs and in swimming pools deeper than 2 m, as well as in diving towers and also in pressurized water chambers. In general, a known prior art equipment, commonly used by divers and scuba divers, is also used in procedures according to the invention to allow underwater breathing and stabilisation of patient's body position.

It is also possible to construct dedicated artificial water reservoirs for one or more users, in which a patient ma lay, sit, swim in a counter-stream, run in place or on treadmill, or else do physical exercises (body bending, limb swinging) under water buoyancy, and using optionally equipment such as parallel bars, horizontal (high) bar, etc. It is also possible to enable visual contact between patients and therapeutists by designing transparent side-walls of water reservoir, as well as manual contact (touch) by designing water reservoir walls equipped at various levels with sleeves and gloves analogous to those employed in laboratory glove-boxes allowing operation in gaseous atmosphere other than air. Regulation and control of hydrostatic pressure in such artificial water reservoirs may be conducted by increasing the heigth of water column (by means of opening of the appropriate valves) to the desired level. When employing corresponding pumps and nozzles allowing swimming in counter-stream, it is necessary to use in the reservoir a closed water circulation system in order to maintain constant height of water column above the patient. A person skilled in the art of water constructions of the type will face no difficulty in providing the reservoirs of the type with fixed infrastructure for breathing and for applying thermal and electric stimuli.

The one-user artificial water reservoir of the type described above designed for prophylactic, physiotherapeutic, rehabilitation or therapeutic procedures has many advantages such as among others: simple construction, relatively low weight and relatively small water volume ensuring beneficial effects of hydrostatic pressure affecting a patient's organism under a total submergence, and in addition a possibility of changing water temperature in the course of individual treatment, a possibility of adjusting the composition of water bath to individual needs of a patient according to patient's health problems, as well as frequent exchange of water in the reservoir, as frequent as even after each use by an individual patient. Also disinfection of such reservoirs is relatively simple, quick and inexpensive.

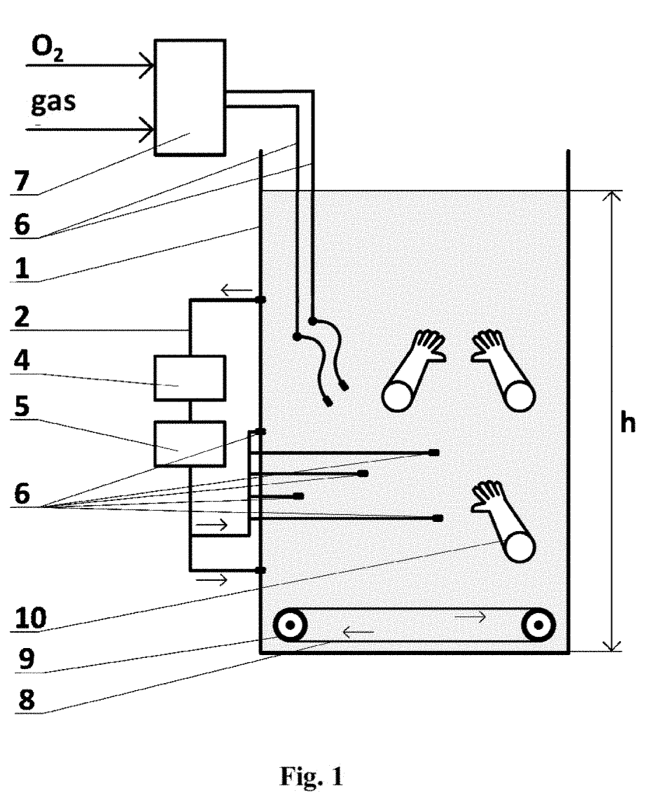

In the accompanying drawing, FIG. 1 presents in a schematic way an exemplary one-user water reservoir for conducting treatments based on the present invention. A reservoir 1 of a height h, allowing generation at the bottom of the reservoir a hydrostatic pressure of up to 2 bar is equipped with a fixed installation 2 for water re-circulation in said reservoir 1, in order to heat or cool it within a heat-exchanger 4 and optionally with pump 5 for creation a counter-stream in the reservoir 1, water being re-circulated to the reservoir 1 via pipes closed with nozzles 3, 3′, 3″, . . . of a adjustable width, and also with a fixed installation 6 delivering gas enabling breathing under the water, the installation including a compressor 7, fed with air and oxygen supplied via separate pipes, which installation introduces to the reservoir 1 from its top the breathing air down to the treatment site and to which both a patient and a therapeutist or accompanying person as mentioned above may be linked via elastic lines ended with gas masks, thus allowing the underwater breathing air, air enriched with oxygen or pure oxygen. At the bottom of the reservoir 1, a treadmill 8 with an external engine 9 may be installed. The inner chamber of reservoir 1 optionally has sleeves with gloves 10, 10′, 10″, . . . installed in sidewalls of the reservoir to enable the therapeutist remaining outside of the inner chamber of the reservoir 1 performing manual procedures or local kinesitherapy (patient's physical exercises) procedures.

### Working Example

A group of 12 healthy volunteers of an age ranging from 35 to 62 years, being at risk of spinal injuries associated with akinetic and sedentary way of their occupational performance, participated in series of procedures consisting of simple physical exercises under water exerting a hydrostatic pressure higher than 1.1 bar, completely surrounding and affecting their organisms, i.e. under total submergence, provided that they worked for at least half a year period after their last holidays. The volunteers were divided into two groups, one of which has operated at the depth of 2.0 m in a diving tower, and the second one—at the depth of 1.5 m in an open swimming pool. The volunteers performed their exercises in a horizontal positions while using for breathing apparatus with compressed air. A control group consisted of 6 further volunteers within the same age range, who did water aerobics in the same swimming pool, without any equipment for the underwater breathing, in vertical position with water level reaching their waists (water ca. 1.1 m deep).

Among the volunteers there was the same number of men and women randomly assigned to the three above mentioned groups.

The participants of the tests were neither diving nor doing water aerobics in the past, although they could swim and were familiar with water environment, also under total submergence.

The series of procedures for the testing groups and for the control group consisted of their participation in 60-minutes exercises, repeated every day, for seven consecutive days.

The after completing the series of test exercises all the participants were surveyed two hours after their last exercises. The participants had to report on their own evaluation—using a scale from 1 to 5—of the change (observed in comparison to the situation preceding the series of test exercises) in the general performance of their organisms, degree of relaxation, improvement of posture, easiness to bend one's back forward from the waist on down and in easiness to squat. The participants could also make other observations on the points not included in the survey.

Despite the relatively low number of participants, significant differences in the results of exercises in the test groups and the control group were noted. Higher scores for individual parameters being evaluated—all values exceeding 2 points—were reported by the participants belonging to the group doing exercises performed under total submergence. In the test groups, among the participants above 50, an improvement of posture and mobility at the level of 3 points was reported by every second volunteer, whereas in the test group exercising at the depth of 2.0 m in a diving tower, all of the volunteers declared a positive change in the general performance of their organisms at the level of 3 to 4 points. Additionally, they have declared reduction of the fatigue experienced after 8 hours of work performed in sitting position. In the control group the lowest score—at the level of 1 point—was reported with respect to change in easiness to bend one's back forward from the waist on down, while the highest score for improvement in the general performance of the participant's organism was 3 points and such a score was reported by one volunteer only. The additional observation reported in the control group related to the swelling reduction at the ankle joints, which earlier used to appear after the whole working day in a sitting position.

The reported results clearly indicate superiority of the treatments carried out under a total submergence at the depth of 1.5 m and 2.0 m, in an environment of water exerting a hydrostatic pressure, completely surrounding and affecting the whole patient's organism under total submergence over the exercises performed in the air, under atmospheric pressure in form of water aerobics, although the observation reported by the volunteer from the control group related to the reduction of swelling at the ankle joints after a day-long work in sitting position, also confirms superiority of the exercises performed under hydrostatic pressure, because during water aerobic exercises the legs of the volunteers were exposed to hydrostatic pressure prevailing at the bottom of the swimming pool at the depth of 1.1 m, for a significant part of each 60-minutes session.

The above description was focused on presentation of general conditions of working the present invention as defined in the appended claims. The invention, however, covers also possible changes and modifications of the described parameters.

1-12. (canceled)

13. A method of prophylactic, physiotherapeutic, rehabilitative and therapeutic treatment of a human patient under total submergence in water completely surrounding and affecting the patient's organism, exerting pressure higher than 1.1 bar, comprising the steps of
a) providing a water reservoir of a depth greater than 1 m, allowing reaching a pressure higher than 1.1 bar;
b) providing an installation or apparatus delivering gas enabling breathing under the water;
c) connecting the patient to the installation or apparatus delivering gas enabling breathing under the water and totally submerging the patient in a vertical or horizontal position under water in the water reservoir, at the depth where the pressure is higher than 1.1 bar;
d) supplying to the patient the air, mixture of gases of a composition different from the composition of the air, such as mixtures of the air and oxygen, or pure oxygen (hyperbaric oxygen therapy HBO/HBOT) for breathing;
e) simultaneously with patient's total submergence, applying selected procedures of classic hydrotherapy, manual therapy, kinesitherapy or combinations thereof as optional procedures additional to the pressure treatment; wherein
duration of the submergence, selection of the breathing gas, selection of additional procedures and the treatment frequency are adjusted to the patient's needs and medical condition(s) to be treated and sufficient to attain desired prophylactic, physiotherapeutic, rehabilitative or therapeutic effect.

14. The method according to claim 13, wherein the water is supplemented with additives appropriate for the type of disorder, such as mineral, gaseous, herbal, anti-inflammatory, anti-microbial—including antibacterial, additives.

15. The method according to claim 13, wherein in step e) local thermal stimuli are used with temperatures higher or lower than patient's body temperature.

16. The method according to claim 13, for treating of disorders, defects and injuries of skeletal system, arthro-skeletal system, musculo-skeletal system and other body systems such as circulatory system, nervous system, immune system or alleviating symptoms thereof.

17. The method according claim 13, for treating or alleviating pain disorders, such as migraine, myalgia and neuralgia.

18. The method according to claim 13, for treating the patients after aesthetic medical treatments, after surgeries, after physical injuries of motoric system.

19. The method according to claim 13, for treating skin diseases, fertility disorders, potency dysfunctions or alleviating symptoms thereof.

20. The method according to claim 13, for treating excess weight, obesity and defects of posture or alleviating symptoms thereof.

21. The method according to claim 13, wherein the pressure applied is preferably higher than 1.2 bar.
